(19) United States
(12) Patent Application Publication
(10) Pub. No.: US 2026/0249011 A1
(43) Pub. Date: Aug. 27, 2026

(54) A SUB-ASSEMBLY OF A MEDICAMENT DELIVERY DEVICE

(71) Applicant: SHL Medical AG, Zug (CH)

(72) Inventor: Pei Yu Chao, Taoyuan City (TW)

(21) Appl. No.: 18/875,780

(22) PCT Filed: Jun. 7, 2023
(86) PCT No.: PCT/EP2023/065181

§ 371 Date: Dec. 17, 2024

(30) Foreign Application Priority Data
Jun. 21, 2022 (EP) 22180140.0

(51) Int. Cl. A61M 5/20; A61M 5/24; A61M 5/32

(52) U.S. Cl. CPC A61M 5/2033; A61M 5/2422; A61M 5/3243

(57) ABSTRACT

The present disclosure provides a sub-assembly of a medicament delivery device. The sub-assembly includes a housing extending along a longitudinal axis between a proximal end of the housing and a distal end of the housing. The sub-assembly also includes a delivery member guard telescopically arranged within the proximal end of the housing. The sub-assembly also includes a plunger rod axially movable along the longitudinal axis within the housing. The sub-assembly also includes a plunger rod driver comprising a flexible portion. The flexible portion is radially flexible relative to the longitudinal axis, and the plunger rod driver engages with the plunger rod and does not axially engage with the delivery member guard when the plunger rod driver is in the distal position. The flexible portion is configured to be flexed radially outward relative to the longitudinal axis by the plunger rod when the plunger rod driver moves from the distal position to the proximal position so that when the plunger rod driver is in the proximal position, the driver body axially disengages from the plunger rod and axially engages with the delivery member guard.

### CROSS-REFERENCE TO RELATED APPLICATIONS

The present application is a U.S. National Phase Application pursuant to 35 U.S.C. § 371 of International Application No. PCT/EP2023/065181 filed Jun. 7, 2023, which claims priority to EP Patent Application No. 22180140.0 filed Jun. 21, 2022. The entire disclosure contents of these applications are herewith incorporated by reference into the present application.

### TECHNICAL FIELD

The present disclosure generally relates to a sub-assembly of a medicament delivery device, and particularly to a sub-assembly of a medicament delivery device with a plunger rod driver.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments such as a growth hormone. Therefore, a medicament delivery device with multiple automatic functions can be desirable as the device can be more user-friendly. However, the cost of manufacturing medicament delivery devices with multiple automatic functions can be high if the numbers of components are high and/or the numbers of metal components are high.

Therefore, a medicament delivery device with few components is desirable.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device. The sub-assembly comprises a housing extending along a longitudinal axis between a proximal end of the housing and a distal end of the housing; a delivery member guard telescopically arranged within the proximal end of the housing; a plunger rod axially movable along the longitudinal axis within the housing. The plunger rod is configured to move into a medicament container. The sub-assembly further comprises a plunger rod driver comprising a flexible portion. The flexible portion is positioned between the housing and the plunger rod in a direction transverse to the longitudinal axis. The flexible portion is radially flexible relative to the longitudinal axis. The plunger rod driver is configured to couple to a power source that is configured to move the plunger rod driver in the direction of the longitudinal axis relative to the housing from a distal position to a proximal position. The plunger rod driver engages with the plunger rod and does not axially engage with the delivery member guard when the plunger rod driver is in the distal position. The flexible portion is configured to be flexed radially outward relative to the longitudinal axis by the plunger rod when the plunger rod driver moves from the distal position to the proximal position so that when the plunger rod driver is in the proximal position, the driver body axially disengages from the plunger rod and axially engages with the delivery member guard.

Preferably, according to another embodiment, the delivery member guard comprises a distally directed surface. The plunger rod comprises a distally directed surface; wherein the flexible portion comprises a first proximally directed surface configured to releasably engage with the distally directed surface of the plunger rod; and wherein the plunger rod driver comprises a second proximally directed surface configured to engage with the distally directed surface of the delivery member guard and a distally directed surface configured to engage with the power source.

Preferably, according to another embodiment, when the plunger rod driver is in the distal position, the first proximally directed surface is adjacent to the distally directed surface of the plunger rod, and the second proximally directed surface is spaced apart from the distally directed surface of the delivery member guard; and when the plunger rod driver is in the proximal position, the first proximally directed surface is disengaged from the distally directed surface of the plunger rod, and the second proximally directed surface is adjacent to the distally directed surface of the delivery member guard.

Preferably, according to another embodiment, the sub-assembly comprises a support surface adjacent to an outer surface of the flexible portion of the driver body in the direction transverse to the longitudinal axis when the driver body is in the proximal position; and the support surface is offset from the outer surface of the flexible portion of the driver body in the direction transverse to the longitudinal axis when the driver body is in the distal position.

Preferably, according to another embodiment, the housing comprises an inner wall extending towards the longitudinal axis; and the support surface is on a surface of the inner wall.

Preferably, according to another embodiment, the flexible portion is radially flexible relative to the longitudinal axis between a relaxed position where the flexible portion flexes radially outward from the longitudinal axis, and a tensioned position where the flexible portion is pressed towards the longitudinal axis by the support surface.

Preferably, according to another embodiment, the flexible portion comprises a pair of flexible arms.

Preferably, according to another embodiment, the pair of flexible arms extends from a fixed, proximal end to a free, distal end in the distal direction of the housing; the first proximally directed surface is arranged at the distal end of the pair of flexible arms.

Preferably, according to another embodiment, the first proximally directed surface is a chamfered surface.

Preferably, according to another embodiment, the sub-assembly comprises the power source and the power source is coupled to the distally directed surface of the driver body.

Preferably, according to another embodiment, the power source comprises a spring.

Preferably, according to another embodiment, the spring is a band spring wound around a pin; the pin is attached to the housing; and the spring comprises a distal end attached to the distally directed surface of the plunger rod driver.

Alternatively, according to another embodiment, the spring is a compression spring extending along the longitudinal axis between a proximal end of the spring and a distal end of the spring; the proximal end of the spring is adjacent to the distally directed surface of the driver body; and the distal end of the compression spring is operably connected to the housing.

Preferably, according to another embodiment, the housing comprises a proximally directed surface adjacent to the distal end of the compression spring.

Alternatively, according to another embodiment, the power source comprises a gas canister.

Alternatively, according to another embodiment, the power source comprises an electric motor.

Preferably, according to another embodiment, the driver body comprises a proximal ledge extending from the driver body in the direction transverse to the longitudinal axis; the distally directed surface of the driver body is defined by a distally directed surface of the proximal ledge and the second proximally directed surface is defined by a proximally directed surface of the proximal ledge.

Preferably, according to another embodiment, a slot or a groove is arranged in a wall of the housing; the slot of the housing or the groove of the housing extends in the direction of the longitudinal axis from a proximal end to a distal end; and the proximal ledge of the driver body is positioned within the slot of the housing or the groove of the housing.

Preferably, according to another embodiment, the delivery member guard comprises a distal body extending from the tubular proximal section of the delivery member guard towards the distal end of the housing; a slot is arranged in the distal body; and the proximal ledge of the driver body is positioned within the slot of the delivery member guard.

Preferably, according to another embodiment, the distally directed surface of the delivery member guard is defined by a proximal edge of the slot of the delivery member guard.

Preferably, according to another embodiment, the plunger rod body is a rod extending along the longitudinal axis between a proximal end of the rod and a distal end of the rod; the proximal end of the rod is configured to contact a distal seal of a medicament container; and the distal end of the rod body defines the distally directed surface of the plunger rod.

Preferably, according to another embodiment, the rod comprises a flange located between the proximal end of the rod and the distal end of the rod in a direction transverse to the longitudinal axis.

Preferably, according to another embodiment, the tubular proximal section of the delivery member guard comprises a fastener and the housing comprises a counter fastener engaged with the fastener; the fastener comprises a distally directed surface; the counter fastener comprises a proximally directed surface; and one of the distally directed surface of the fastener and the counter fastener of the housing is a chamfered surface.

Preferably, according to another embodiment, one of the fastener and the counter fastener is a protrusion and the other one of the fastener and the counter fastener is a recess or a cut-out.

Preferably, according to another embodiment, the sub-assembly comprises a rotator; the housing comprises a distally directed surface; the rotator comprises a proximally directed surface adjacent to the distally directed surface of the housing and a distally directed surface; the driver body of the plunger rod driver comprises a third proximally directed surface; the rotator is rotatable around the longitudinal axis relative to the plunger rod driver between a first position where the distally directed surface of the rotator is adjacent to the third proximally directed surface of the driver body and a second position where the distally directed surface of the rotator is circumferentially offset from the third proximally directed surface of the driver body.

Preferably, according to another embodiment, the rotator comprises a user-accessible section extending out from the housing; and the rotator is configured to be rotated by a user from the first position to the second position.

Alternatively, according to another embodiment, the rotator comprises a proximally directed helical surface extending from a proximal end to a distal end; the distal body of the delivery member guard comprises a distally directed surface configured to move along the proximally directed helical surface of the rotator when the delivery member guard is moved towards the distal end of the housing.

Alternatively, according to another embodiment, the sub-assembly comprises a distal button extending out from the distal end of the housing; the distal button is axially movable relative to the housing; the rotator comprises a distally directed helical surface extending from a proximal end of the rotator to a distal end of the rotator; the distal button comprises a button body extending into the housing; the button body comprises a distally directed surface configured to move along the distally directed helical surface of the rotator when the distal button is moved towards the proximal end of the housing.

Preferably, according to another embodiment, the rotator comprises a tubular body surrounding the flexible portion of the plunger rod driver when the plunger rod driver is in the distal position. Preferably, according to another embodiment, the support surface is defined by an inner surface of the tubular body of the rotator.

Preferably, according to another embodiment, a cut-out is arranged in a wall of the driver body of the plunger rod driver; the third proximally directed surface of the driver body is next to the cut-out; the rotator comprises a proximally directed arm positioned through the cut-out of the driver body when the plunger rod driver is in the distal position; and the proximally directed arm comprises a ledge extending from a proximal end of the proximally directed arm in the direction transverse to the longitudinal axis; and the distally directed surface of the rotator is defined by the ledge.

Preferably, according to another embodiment, the medicament delivery device comprises a multiple-chamber medicament container arranged within the carrier.

Preferably, according to another embodiment, the medicament delivery device is an auto-injector or a manual insulin pen.

Preferably, according to another embodiment, the medicament delivery device is an auto-injection with only one spring.

Preferably, according to another embodiment, the medicament container is a medicament container without a medicament delivery member.

Preferably, according to another embodiment, the medicament container is a glass cartridge, a plastic cartridge, or a collapsible bag.

Preferably, according to another embodiment, the medicament delivery member is an injection needle, an infusion catheter, or a spray nozzle.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives.

Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a sub-assembly of the invention.

FIG. 2 schematically shows a perspective view of components of the sub-assembly of FIG. 1.

FIG. 3 schematically shows a perspective view of a plunger rod drive with a power source of the sub-assembly of FIG. 2.

FIG. 4 schematically shows a perspective view of the plunger rod driver of FIG. 3.

FIG. 5 schematically shows a perspective view of the plunger rod driver and a delivery member guard of FIG. 3.

FIG. 6 schematically shows a perspective view of the plunger rod driver, the delivery member guard and the plunger rod of FIG. 3.

FIG. 7 schematically shows a perspective view of a rotator of FIG. 3.

FIG. 8 schematically shows a perspective view of the rotator and the plunger rod driver of FIG. 3.

FIG. 9 schematically shows a cross-section view of the sub-assembly of FIG. 1 when the plunger rod drive is in a distal position.

FIG. 10 schematically shows a cross-section view of the sub-assembly of FIG. 1 when the plunger rod drive is in a proximal position.

FIG. 11 schematically shows a perspective view of the plunger rod driver of FIG. 3.

FIG. 12 schematically shows a perspective view of the plunger rod driver and the plunger rod of FIG. 3 and a medicament container.

FIGS. 13-15 schematically show perspective views of the sub-assembly of FIG. 1.

FIG. 16 schematically shows a perspective view of a housing of FIG. 3.

FIG. 17 schematically shows a perspective view of a medicament container carrier of FIG. 3.

FIG. 18 schematically shows a perspective view of the medicament container carrier of FIG. 17 and the medicament container.

FIGS. 19-20 schematically show perspective views of the sub-assembly of FIG. 1.

### DETAILED DESCRIPTION

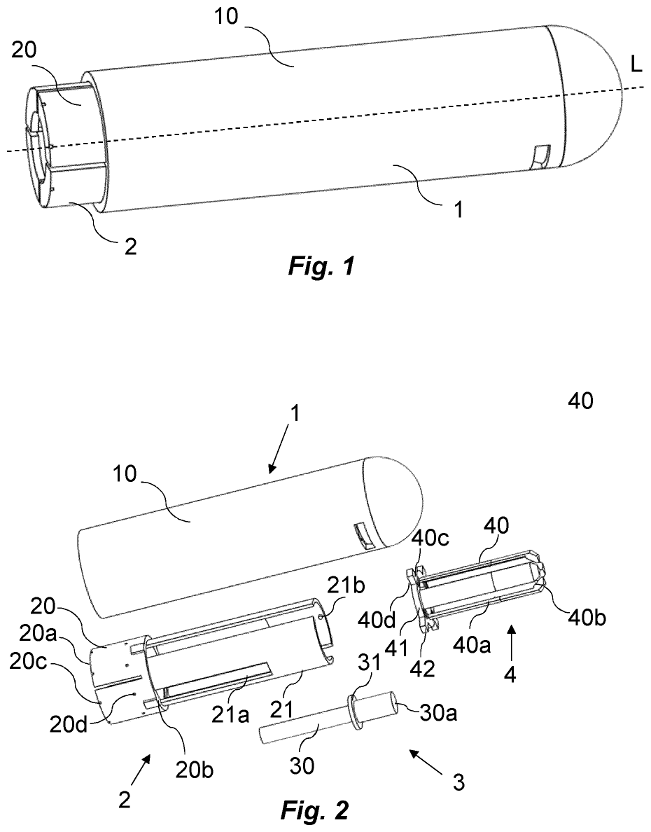

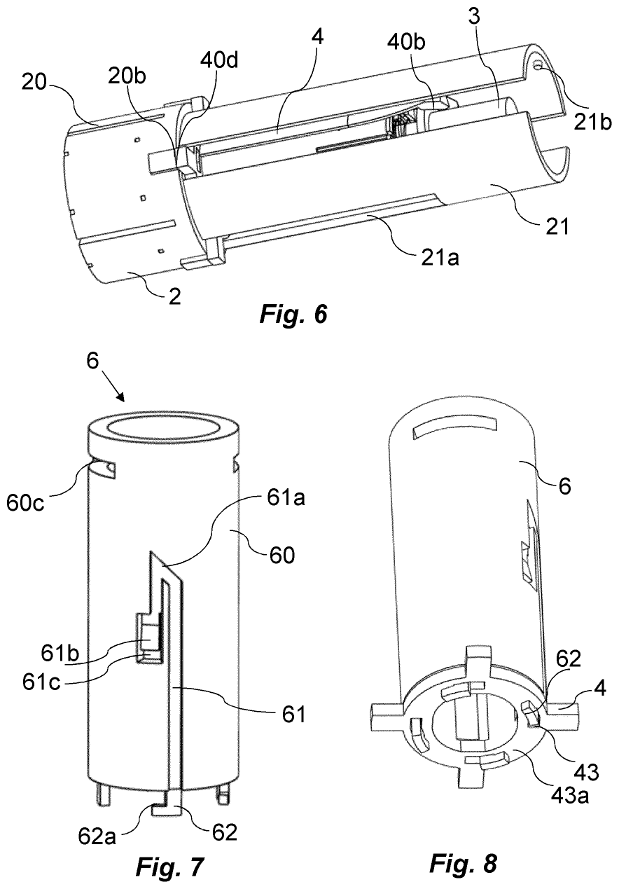

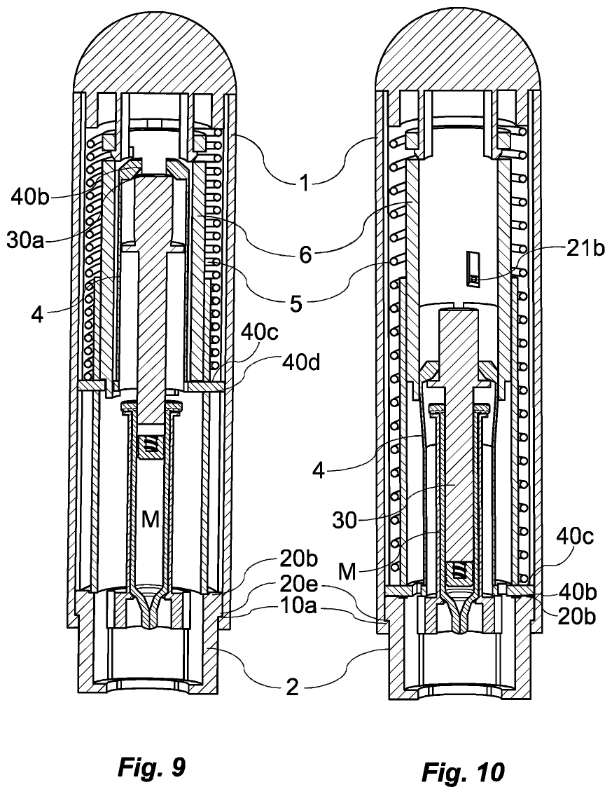

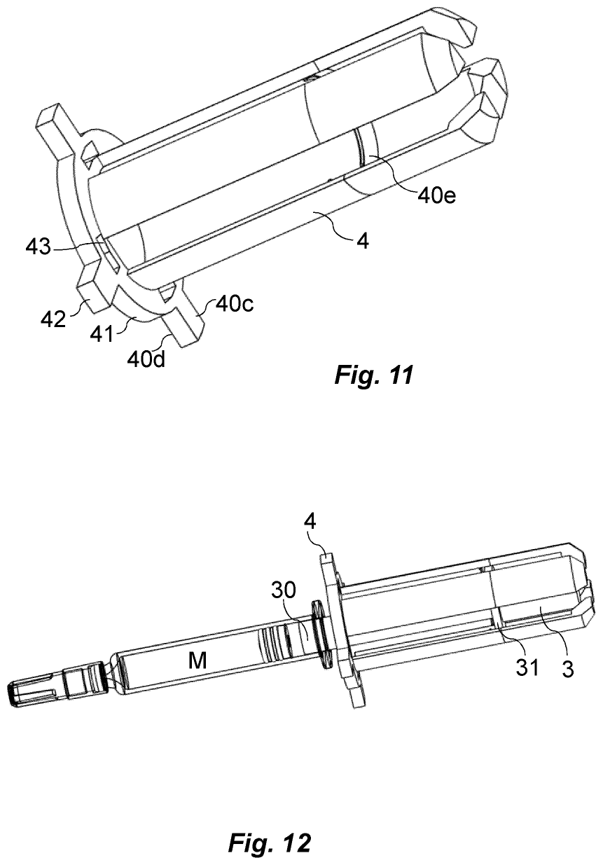

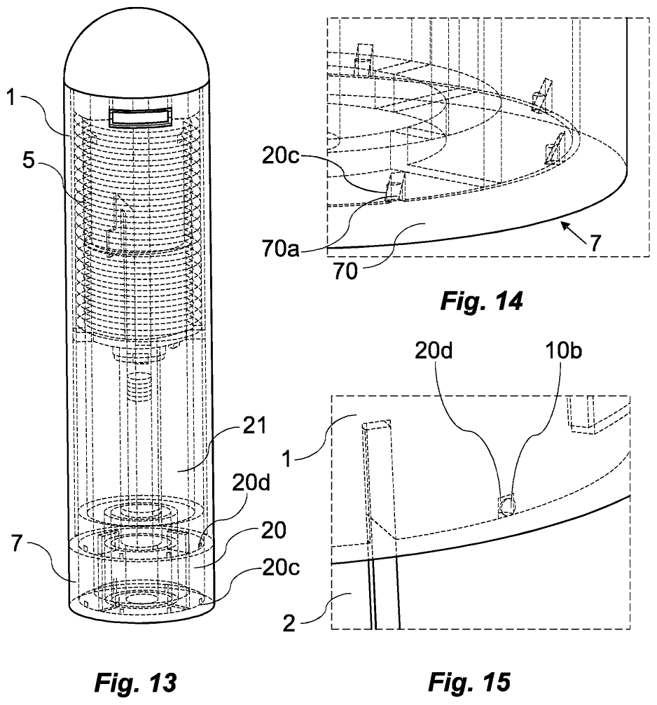

FIGS. 1-20 illustrate a sub-assembly of a medicament delivery device. The sub-assembly comprises a housing 1, a delivery member guard 2, a plunger rod 3, and a plunger rod driver 4. The housing 1 extends along a longitudinal axis L between a proximal end of the housing 1 and a distal end of the housing 1.

The housing 1 comprises a body 10. The body 10 is configured to accommodate a medicament container. In a preferred example, the body 10 is matched with the shape of the medicament container that is configured to be received within the body 10. For example, if the medicament container is generally cylindrical, e.g., a syringe or a cartridge, the body 10 of the housing 1 can be also generally cylindrical, as shown in FIG. 1, so that the device can be compact. Alternatively, if the medicament container is a collapsible bag, e.g., generally flat, the body of the housing can be generally flat and have rectangular side surfaces. Alternatively, the body of the housing can be in other suitable shapes dependent on the design, e.g., considering particular target patient groups.

The delivery member guard 2 is telescopically arranged within the proximal end of the housing 1, as shown in FIG. 1. The delivery member guard 2 comprises a tubular proximal body 20 configured to surround a medicament delivery member, e.g., a needle or a nozzle. The tubular proximal 20 comprises a proximal end wall 20a configured to be in contact with a medicament delivery site, e.g., a patient's skin. The delivery member guard 2 comprises a distal body 21 extending from the tubular proximal body 20 into the body 10 of the housing 1. In one example, the distal body 21 comprises a distally extending arm 21. The delivery member guard 2 comprises a distally directed surface 20b. In one example, the distally directed surface 20b of the delivery member guard 2 is defined by a distal end of the tubular proximal body 20, e.g., a distal end edge of the tubular proximal body, as shown in FIG. 2. Alternatively, the distally directed surface is defined by a protrusion extending from the distal body towards the longitudinal axis L. Optionally, the tubular proximal body 20 of the delivery member guard 2 comprises a fastener 20d and the housing 1 comprises a counter fastener 10b engaged with the fastener. In a preferred example, the fastener 20d comprises a distally directed surface and the counter fastener 10b comprises a proximally directed surface. In a preferred example, one of the distally directed surface of the fastener 20d and the counter fastener 10b of the housing is a chamfered surface. In another preferred example, one of the fastener 20d and the counter fastener 10b is a protrusion and the other one of the fastener 20d and the counter fastener 10b is a recess or a cut-out. In a preferred example, the fastener 20d is arranged on the proximal tubular body 20 of the delivery member guard 2. Alternatively, the fastener 20d is arranged on the distal body of the delivery member guard. As the sub-assembly is configured to transfer a force from a power source to the delivery member guard 2 after a predetermined amount of medicament is delivered (as will be explained later), the engagement between the fastener 20d of the delivery member guard 2 and the counter fastener 10b of the housing 1 is configured to prevent the delivery member guard 2 from moving in the distal direction of the housing 1 before use of the medicament delivery device. In one example, the fastener 20d is a recess, and the counter fastener 10b is a protrusion, as shown in FIG. 15. In a preferred example, the recess and/or the protrusion comprises a chamfered surface such that the abutment between the fastener 20d and the counter fastener 10b can be overcome when the delivery member guard 2 is moved in the distal direction by force above a threshold. The threshold can be designed by the inclination of the chamfer surface and/or the material flexibility of the fastener and the counter fastener. For example, the protrusion 10b can be made of resilient material.

This engagement can be removed from the design for the device without a preassembled medicament delivery member, e.g., a device that the user needs to attach a medicament delivery member before use. In other words, for some medicament delivery device, the delivery member guard doesn't need to be prevented from being moved before use of the medicament delivery device. For example, some medicament delivery devices do not have needles at the beginning, e.g., the user needs to attach a pen needle onto the medicament delivery device before use, or some medicament delivery devices do not have a needle at all, e.g., a jet injector. For those medicament delivery devices, the delivery member guard 2 doesn't need to protrude from the housing before use. However, as the delivery member in this example, e.g., the pen needle and/or the spray nozzle of the jet injector, might contact blood or any other body fluid during medicament delivery operation, a used delivery member needs to be shielded.

In another example, the proximal tubular body 20 of the delivery member guard 2 comprises a cap fastener 20c. In this example, the sub-assembly comprises a cap 7 comprising a cap body 70 and a counter cap fastener 70a. The counter cap fastener 70a is configured to releasably engage with the cap fastener 20c of the delivery member guard 2. The cap body 70 is configured to cover the proximal end of the delivery member guard 2.

The plunger rod 3 is axially movable along the longitudinal axis L within the housing 1. The plunger rod 3 is configured to move into a medicament container M. The plunger rod comprises a rod body 30. The rod body 30 extends along the longitudinal axis L between a proximal end of the rod body and a distal end of the rod body; wherein the proximal end of the rod is configured to contact a distal seal of a medicament container.

In a preferred example, the rod body is tubular so that the material for making the rod body can be reduced. Alternatively, the rod body can be a solid core body so that the plunger rod 3 can bear a high force. The plunger rod 3 comprises a distally directed surface 30a. Preferably, the distally directed surface 30a is defined by the distal end of the rod body 30 of the plunger rod 3, as shown in FIG. 2. Alternatively, the distally directed surface is defined by a ledge extending from the rod body in a direction transverse to the longitudinal axis L. In a preferred example, the plunger rod comprises a flange 31 extending from the rod body in a direction transverse to the longitudinal axis L, as shown in FIG. 2.

The plunger rod driver 4 comprises a driver body 40 having a flexible portion 40a. The flexible portion 40a is positioned between the housing 1 and the plunger rod 3 in the direction transverse to the longitudinal axis L. The flexible portion 40a is radially flexible relative to the longitudinal axis L. The plunger rod driver 4 is configured to connect with a power source 5 that is configured to move the plunger rod driver 4 in the direction of the longitudinal axis relative to the housing 1 from a distal position to a proximal position. The plunger rod driver 4 engages with the plunger rod 3 and does not axially engage with the delivery member guard 2 when the plunger rod driver 4 is in the distal position. The flexible portion 40a is configured to be flexed radially outward relative to the longitudinal axis L by the plunger rod 3 when the plunger rod driver 4 moves from the distal position to the proximal position so that when the plunger rod driver 4 is in the proximal position, the driver body 40 axially disengages from the plunger rod 3 and axially engages with the delivery member guard 2. The sub-assembly can also provide an end indication. In one example where the delivery member guard 2 is configured to be in contact with the medicament delivery site, e.g., when the medicament delivery device comprising the sub-assembly is an auto-injector, in this example, when the driver body 40 axially engaged with the delivery member guard 2, the user gets a tactile indication on an injection site as the delivery member guard 2, in this example, is configured to contact the injection site, so that an end of use indication is provided to the user.

The flexible portion 40a comprises a first proximally directed surface 40b configured to releasably engage with the distally directed surface 30a of the plunger rod 3. The plunger rod driver 4 comprises a second proximally directed surface 40d configured to engage with the distally directed surface 20b of the delivery member guard 2 and a distally directed surface 40c configured to couple to the power source 5, as shown in FIG. 4. In one example, the flexible portion 40a comprises a pair of flexible arms 40a. In a preferred example, the pair of flexible arms 40a extends from a fixed, proximal end to a free, distal end in the distal direction of the housing 1. The first proximally directed surface 40b is arranged at the distal end of the pair of flexible arms 40a, as shown in FIG. 4. Alternatively, the driver body is a resilient sleeve, e.g., a sleeve made of resilient material. In this example, the first proximally directed surface is defined by a ledge extending from an inner surface of the resilient sleeve. In a preferred example, the first proximally directed surface 40b is a chamfered surface.

In a preferred example, the driver body 40 comprises a proximal ledge 41 extending from the driver body 40 in the direction transverse to the longitudinal axis L. In a preferred example, the distally directed surface 40c of the plunger rod driver 40 is defined by a distally directed surface 40c of the proximal ledge 41. In another example, the second proximally directed surface 40d is defined by a proximally directed surface of the proximal ledge 41. Alternatively, the second proximally directed surface is defined by the proximal end of the driver body, and the distally directed surface 40c is defined by a ledge extending from the driver body.

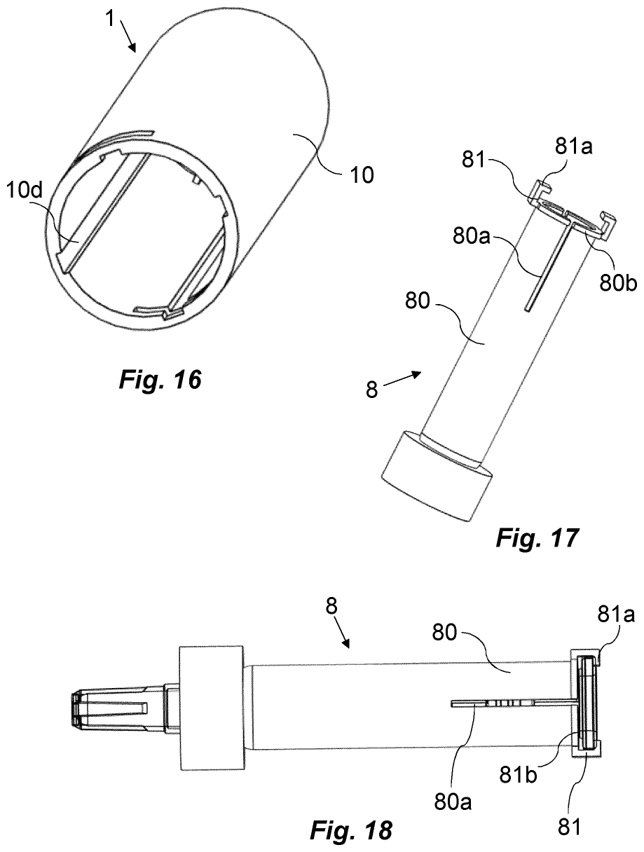

In a preferred example, a groove 10d is arranged in a wall of the housing 1, as shown in FIG. 16. Alternatively, the groove 10d can also be a slot. The groove of the housing 1 extends in the direction of the longitudinal axis L from a proximal end to a distal end. In this example, the proximal ledge 41 of the driver body 40 is positioned within the groove 10d of the housing 1. The groove 10d provides an assembling guide so that the driver body can be easily assembled into the housing.

In another example, a slot 21a is arranged in the distal body 21 of the delivery member guard 2, as shown in FIG. 5. In this example, the proximal ledge 41 of the driver body 40 is positioned within the slot 21a of the delivery member guard 2. In this example, the distally directed surface 20b of the delivery member guard 2 is defined by a proximal edge of the slot 21a of the delivery member guard 2.

When the plunger rod driver 4 is in the distal position, the first proximally directed 40b surface is adjacent to the distally directed surface 30a of the plunger rod 3, and the second proximally directed surface 40d is spaced apart from the distally directed surface 20b of the delivery member guard 2. When the plunger rod driver 4 is in the proximal position, the first proximally directed surface 40a is disengaged from the distally directed surface 30a of the plunger rod 3, and the second proximally directed surface 40d is adjacent to the distally directed surface 20a of the delivery member guard 2. The exact position of the proximal position of the plunger rod driver 4 and the distal position of the plunger rod driver 4 are dependent on the design of the medicament delivery device. For example, if a medicament delivery device is designed to empty all containing medicament, the plunger rod 3 is configured to disengage from the plunger rod driver 4 when the plunger rod 3 cannot move any further within the medicament container. In this example, as the plunger rod cannot move any further within the medicament container, the reaction force from the distally directed surface 30a of the plunger rod 3 applies on the first proximally directed 40b surface of the plunger rod driver 4 radially deforms the flexible portion 40a of the plunger rod driver 4. Therefore, the first proximally directed surface 40a is disengaged from the distally directed surface 30a of the plunger rod 3. The plunger rod driver 4 is continuously moved by the power source, and thus, the second proximally directed surface 40d is moved towards the distally directed surface 20a of the delivery member guard 2 until the second proximally directed surface 40d is adjacent to the distally directed surface 20a of the delivery member guard 2. Therefore, the plunger rod driver 4 is in the distal position. When the plunger rod driver 4 is in the distal position, the force from the power source is transferred to the delivery member guard 2 and thereby the delivery member guard 2 is moved to a final, after-use position to cover a used medicament delivery member.

Optionally, the sub-assembly comprises a support surface 60a adjacent to an outer surface of the flexible portion 40a of the driver body 40 in the direction transverse to the longitudinal axis L when the driver body is in the proximal position. The support surface 60a is offset from the outer surface of the flexible portion 40a of the driver body 40 in the direction transverse to the longitudinal axis L when the driver body 40 is in the distal position. In this example, the distal position of the plunger rod driver 4 is determined by the location of the support surface. For example, the support surface enables the plunger rod 3 to engage with the plunger rod driver 4 when the plunger rod 3 has already emptied the medicament container so that a potential drooling issue due to the residual medicament, e.g., medicament that is trapped within the medicament delivery member or that is trapped by the surface tension force of the medicament container, can be avoided.

In a preferred example, the housing comprises an inner wall extending towards the longitudinal axis, and the support surface is on a surface of the inner wall.

In another preferred example, the flexible portion 40a is radially flexible relative to the longitudinal axis L between a relaxed position where the flexible portion 40a flexes radially outward from the longitudinal axis L and a tensioned position where the flexible portion 40a is pressed towards the longitudinal axis L by the support surface 60a.

When the plunger rod driver 4 is in the distal position, the force from the power source is transferred to the delivery member guard 2, and thus, the delivery member guard 4 can be moved in the proximal direction relative to the housing 1 and cover the medicament delivery member. In one example, the housing comprises one or more ribs to act as a proximal stop for the plunger rod driver 4 and/or the medicament delivery guard 2, so that when the medicament delivery member guard 2 is blocked by the proximal stop (directly or indirectly via the plunger rod driver 4), the medicament delivery member guard 2 and the plunger rod driver 4 cannot move axially relative to the housing in the proximal direction of the housing. Alternatively, a medicament container carrier of the medicament delivery device can provide a proximal stop for the plunger rod driver 4 and/or the medicament delivery guard 2.

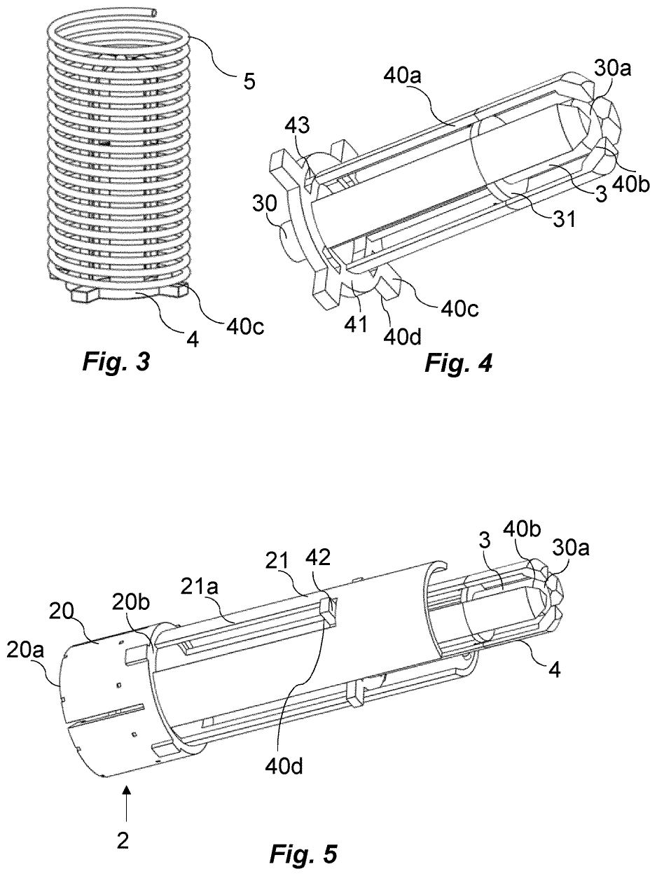

In a preferred example, the power source comprises a spring 5. The spring can be a band spring or a compression spring. In an example, as shown in FIG. 3, the spring is a compression spring extending along the longitudinal axis L between a proximal end of the spring and a distal end of the spring. The proximal end of the spring 5 is adjacent to the distally directed surface 40c of the driver body 40. The distal end of the compression spring is operably connected to the housing, e.g., directly or indirectly engaged with the housing. In a preferred example, the housing comprises a proximally directed surface adjacent to the distal end of the compression spring. In another example, the sub-assembly comprises the power source 5 and the power source 5 is coupled to the distally directed surface of the driver body 4. In one example, the power source is a gas canister with a valve. In this example, when the power source is activated, the gas is released, with the result that the gas pushes on the distally directed surface of the driver body. In another example, the power source is a motor and a gear set. In this example, the gear set comprises a shaft engaging with the distally directed surface of the driver body.

In another example, the sub-assembly comprises a rotator 6, as shown in FIGS. 7-8. The rotator 6 comprises a body 60. In one example, the body 60 of the rotator 6 is tubular. Alternatively, the body of the rotator can be a clip or semi-cylindrical.

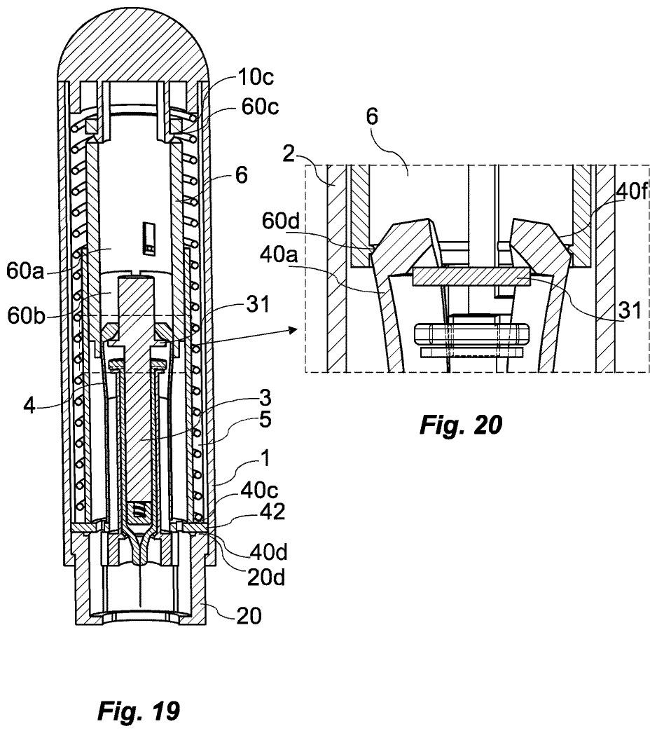

The housing 1 comprises a distally directed surface 10c and the rotator 6 comprises a proximally directed surface 60c adjacent to the distally directed surface 10c of the housing 1 (as shown in FIG. 19) and a distally directed surface 62a.

The rotator 6 is configured to hold the plunger rod driver 4 so that the plunger rod driver 4 is prevented from moving axially relative to the housing 1. The rotator 6 is configured to be rotated by a trigger about the longitudinal axis to disengage from the plunger rod driver 4, thereby releasing the plunger rod driver 4 from the housing 1.

In one example, the trigger is the delivery member guard 2. In this example, the rotator 6 comprises a proximally directed helical surface 61c extending from a proximal end to a distal end. In this example, the distal body 21 of the delivery member guard 2 comprises a distally directed surface 21b configured to move along the proximally directed helical surface 61c of the rotator 6 when the delivery member guard 2 is moved towards the distal end of the housing 1. Therefore, the rotator 6 is rotated to disengage from the plunger rod driver 4.

In one example, the rotator 6 comprises a distally directed surface 62a, and the driver body 40 of the plunger rod driver 4 comprises a third proximally directed surface 43a. The rotator 6 is rotatable around the longitudinal axis L relative to the plunger rod driver 4 between a first position where the distally directed surface 62a of the rotator 6 is adjacent to the third proximally directed surface 43a of the driver body 40 and a second position where the distally directed surface 61a of the rotator 6 is circumferentially offset from the third proximally directed surface 43a of the driver body 40. In a preferred example, the rotator comprises a proximally extending arm 62, the distally directed surface 62a is defined at the proximal end of the arm 62. In another example, and a cut-out is arranged in the proximal ledge 41 of the driver body 40 of the plunger rod driver 4. In this example, the third proximally directed surface 43a is defined by the proximal ledge 41. The third proximally directed surface 43a of the driver body 40 is next to the cut-out.

In this example, the rotator 6 comprises the proximally directed arm 62 positioned through the cut-out of the driver body 40 when the plunger rod driver 4 is in the distal position. In this example, the proximally directed arm 62 comprises a ledge extending from a proximal end of the proximally directed arm 62 in the direction transverse to the longitudinal axis L and the distally directed surface 62a of the rotator 6 is defined by the ledge, as shown in FIG. 8.

It should be noted that instead of the delivery member guard 2 acting as the trigger, the trigger can be a distal button extending out from the distal end of the housing. In this example, the distal button is axially movable relative to the housing. In this example, the rotator comprises a distally directed helical surface extending. The distal button comprises a button body extending into the housing. The button body comprises a distally directed surface configured to move along the distally directed helical surface of the rotator when the distal button is moved towards the proximal end of the housing.

Alternatively, the rotator comprises a user-accessible section extending out from the housing. In this example, the rotator is configured to be rotated by a user from the first position to the second position.

The rotator can be involved in the mechanism that directly triggers the medicament delivery operation. For example, when the power source is a spring, the rotator can be configured to hold the plunger rod driver 4 until the rotator is rotated to disengage the plunger rod driver 4, thereby causing the plunger rod driver 4 and the plunger rod 3 to move in the proximal direction relative to the housing. Additionally, the rotator can be a safety mechanism. For example, when the power source is a set of motor gears or when the power source comprises a gas canister, the rotator is a safety mechanism that prevents a user from unintentionally triggering the medicament delivery operation. For example, the user might need to manually rotate the user-accessible section of the rotator to disengage the rotator from the plunger rod driver, so that when the motor is switched on or the gas is released, the plunger rod driver and the plunger rod can move relative to the housing.

In one example where the sub-assembly comprises the rotator 6 and the rotator 6 comprises the tubular body 60, the tubular body 60 optionally surrounds the flexible portion 40a of the plunger rod driver 4 when the plunger rod driver 4 is in the distal position. In this example, the support surface 60a is defined by an inner surface of the tubular body 60 of the rotator 6.

In another example, the rotator 6 comprises a lockout tab 61b that defines a proximally directed surface 61c configured to block a distally directed surface of the delivery member guard 2 so that the delivery member guard 2 cannot move axially in the distal direction relative to the housing 1 when the delivery member guard 2 is in the final, after-use position.

In one example, the plunger rod driver 4 comprises a support ledge 40e configured to engage the flange 31 of the plunger rod 3. The support ledge 40e can be a guide for guiding the assembling step for assembling the plunger rod 3 into the plunger rod driver 4.

In another example, the flange 31 of the plunger rod 3 is configured to further deform the flexible portion 40a of the plunger rod driver 4 when the delivery member guard 2 is in the final, after-use position, as shown in FIGS. 19-20. In this example, the plunger rod driver comprises a second distally directed surface 40f extending from the flexible portion 40a of the plunger rod driver 4. In one example where the sub-assembly comprises the rotator 6, the rotator 6 comprises a ledge 60d extending from the body 60 of the rotator 6 towards the plunger rod driver 4. When the flange 31 of the plunger rod 3 further deforms the flexible portion 40a, the second distally directed surface 40f abuts a proximally directed surface that is defined by the ledge 60d of the rotator 6. This feature can act as a delivery member guard lockout mechanism as the plunger rod driver 4 is blocked from moving in the distal direction of the housing 1 by the rotator 6, with the result that the delivery member guard 2 is blocked from moving in the distal direction of the housing 1. In this example, the lockout tab of the rotator can be removed. Alternatively, this feature can be a secondary safety mechanism that prevents the plunger rod 3 from moving in the distal direction of the housing 1. As the flange 31 of the plunger rod 3 is engaged with the flexible portion 40a of the plunger rod driver 4 and the distal movement of the plunger rod driver 4 is blocked, the plunger rod 3 cannot move in the distal direction relative to the housing 1.

In another preferred example, the sub-assembly comprises a container carrier 8, as shown in FIG. 17. The container carrier 8 comprises a body 80 configured to accommodate a medicament container, e.g., a syringe or a cartridge. In one example, the body 80 is tubular. In one example, the body 80 extends along the longitudinal axis L from a proximal end of the body to a distal end of the body. In this example, a slot 80a extends from the distal end of the body 80 towards the proximal end of the body 80. In a preferred example, the slot 80a extends no further than half of the entire length of the body as measured from the distal end of the body to the proximal end of the body. Therefore, the distal end of the body can flex radially outwardly relative to the longitudinal axis during an insertion of a medicament container into the body 80. In one example where the medicament container is a syringe, the distal end 80b of the body 80 is configured to support a proximal surface of a flange of the syringe. In a preferred example, the container carrier 8 comprises a distally extended arm 81 with a transverse ledge 81a at its free end. The transverse ledge 81a is configured to support a distal surface of the flange of the syringe.

Therefore, the syringe is prevented from moving axially relative to the container carrier 8. In a preferred example, an inner surface of the distally extended arm 81 abuts the flange of the syringe in the direction transverse to the longitudinal axis L, therefore, the syringe is prevented from radially rattling relative to the container carrier 8.

The container carrier 8 is configured to be arranged within the housing 1 and is configured to be attached to the housing. In a preferred example, the container carrier 8 is used when the housing 1 accommodates a small size medicament container. For example, the housing 1 can be designed to directly accommodate a syringe that is configured to contain 3 mL of medicament. In this example, the syringe can be attached to the housing 1 directly without using a container carrier. However, when a small size syringe, e.g., a syringe that is configured to contain 1 mL of medicament, is used, the small size syringe can be directly accommodated within the container carrier 8, thereby the small size syringe can be attached to the housing via the container carrier 8. Therefore, the housing 1 can be standardized for medicament containers of different sizes.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are described in the clauses below.
1. A sub-assembly of a medicament delivery device, the sub-assembly comprising:
a housing (1) extending along a longitudinal axis (L) between a proximal end of the housing and a distal end of the housing;
a delivery member guard (2) telescopically arranged within the proximal end of the housing (1);
a plunger rod (3) axially movable along the longitudinal axis (L) within the housing (1), wherein the plunger rod (3) is configured to move into a medicament container (M); a plunger rod driver (4) comprising a driver body (40) having a flexible portion (40a);
wherein the flexible portion (40a) is positioned between the housing (1) and the plunger rod (3) in a direction transverse to the longitudinal axis (L); wherein the flexible portion (40a) is radially flexible relative to the longitudinal axis (L);
wherein the plunger rod driver (4) is configured to couple to a power source (5) that is configured to move the plunger rod driver (4) in the direction of the longitudinal axis relative to the housing (1) from a distal position to a proximal position;
wherein the plunger rod driver (4) engages with the plunger rod (3) and does not axially engage with the delivery member guard (2) when the plunger rod driver (4) is in the distal position; and
wherein the flexible portion (40a) is configured to be flexed radially outward relative to the longitudinal axis (L) by the plunger rod (3) when the plunger rod driver (4) moves from the distal position to the proximal position so that when the plunger rod driver (4) is in the proximal position, the driver body (40) axially disengages from the plunger rod (3) and axially engages with the delivery member guard (2).
2. The sub-assembly according to clause 1, wherein the delivery member guard comprises a distally directed surface; wherein the plunger rod comprises a distally directed surface; wherein the flexible portion comprises a first proximally directed surface configured to releasably engage with the distally directed surface of the plunger rod; and wherein the plunger rod driver comprises a second proximally directed surface configured to engage with the distally directed surface of the delivery member guard and a distally directed surface configured to engage with the power source.
3. The sub-assembly according to clause 2, wherein when the plunger rod driver is in the distal position, the first proximally directed surface is adjacent to the distally directed surface of the plunger rod, and the second proximally directed surface is spaced apart from the distally directed surface of the delivery member guard; and wherein when the plunger rod driver is in the proximal position, the first proximally directed surface is disengaged from the distally directed surface of the plunger rod, and the second proximally directed surface is adjacent to the distally directed surface of the delivery member guard.
4. The sub-assembly according to any one of the preceding clauses, wherein the sub-assembly comprises a support surface (60a) adjacent to an outer surface of the flexible portion (40a) of the driver body (40) in the direction transverse to the longitudinal axis (L) when the driver body is in the proximal position; and wherein the support surface (60a) is offset from the outer surface of the flexible portion (40a) of the driver body (40) in the direction transverse to the longitudinal axis (L) when the driver body (40) is in the distal position.
5. The sub-assembly according to clause 4, wherein the housing comprises an inner wall extending towards the longitudinal axis; and wherein the support surface is on a surface of the inner wall.
6. The sub-assembly according to clause 4 or 5, wherein the flexible portion is radially flexible relative to the longitudinal axis between a relaxed position where the flexible portion flexes radially outward from the longitudinal axis, and a tensioned position where the flexible portion is pressed towards the longitudinal axis by the support surface.
7. The sub-assembly according to any of the preceding clauses, wherein the flexible portion comprises a pair of flexible arms.
8. The sub-assembly according to clause 7, wherein the pair of flexible arms extends from a fixed, proximal end to a free, distal end in the distal direction of the housing; wherein the first proximally directed surface is arranged at the distal end of the pair of flexible arms.
9. The sub-assembly according to any one of the preceding clauses, wherein the first proximally directed surface is a chamfered surface.
10. The sub-assembly according to clause 9, wherein the power source comprises a spring.
11. The sub-assembly according to clause 10, wherein the spring is a compression spring extending along the longitudinal axis between a proximal end of the spring and a distal end of the spring; wherein the proximal end of the spring is adjacent to the distally directed surface of the driver body; and wherein the distal end of the compression spring is operably connected to the housing.
12. The sub-assembly according to clause 11, wherein the housing comprises a proximally directed surface adjacent to the distal end of the compression spring.
13. The sub-assembly according to any one of the preceding clauses, wherein the driver body comprises a proximal ledge extending from the driver body in the direction transverse to the longitudinal axis; wherein the distally directed surface of the plunger rod driver is defined by a distally directed surface of the proximal ledge and the second proximally directed surface is defined by a proximally directed surface of the proximal ledge.
14. The sub-assembly according to clause 13, wherein a slot or a groove is arranged in a wall of the housing; wherein the slot of the housing or the groove of the housing extends in the direction of the longitudinal axis from a proximal end to a distal end; and wherein the proximal ledge of the driver body is positioned within the slot of the housing or the groove of the housing.
15. The sub-assembly according to clause 13 or 14, wherein the delivery member guard comprises a distal body extending from the tubular proximal body of the delivery member guard towards the distal end of the housing; wherein a slot is arranged in the distal body; and wherein the proximal ledge of the driver body is positioned within the slot of the delivery member guard.
16. The sub-assembly according to clause 15, wherein the distally directed surface of the delivery member guard is defined by a proximal edge of the slot of the delivery member guard.
17. The sub-assembly according to any one of the preceding clauses, wherein the plunger rod comprises a rod body extending along the longitudinal axis between a proximal end of the rod body and a distal end of the rod body; wherein the proximal end of the rod body is configured to contact a distal seal of a medicament container; and wherein the distal end of the rod body defines the distally directed surface of the plunger rod.
18. The sub-assembly according to clause 17, wherein the rod comprises a flange located between the proximal end of the rod and the distal end of the rod in a direction transverse to the longitudinal axis.
19. The sub-assembly according to any one of the preceding clauses, wherein the tubular proximal body of the delivery member guard comprises a fastener and the housing comprises a counter fastener engaged with the fastener; wherein the fastener comprises a distally directed surface; wherein the counter fastener comprises a proximally directed surface; and wherein one of the distally directed surface of the fastener and the counter fastener of the housing is a chamfered surface.
20. The sub-assembly according to clause 19, wherein one of the fastener and the counter fastener is a protrusion and the other one of the fastener and the counter fastener is a recess or a cut-out.
21. The sub-assembly according to any one of the preceding clauses, wherein the sub-assembly comprises a rotator; wherein the housing comprises a distally directed surface; wherein the rotator comprises a proximally directed surface adjacent to the distally directed surface of the housing and a distally directed surface; wherein the driver body of the plunger rod driver comprises a third proximally directed surface; wherein the rotator is rotatable around the longitudinal axis relative to the plunger rod driver between a first position where the distally directed surface of the rotator is adjacent to the third proximally directed surface of the driver body and a second position where the distally directed surface of the rotator is circumferentially offset from the third proximally directed surface of the driver body.
22. The sub-assembly according to clause 21, wherein the rotator comprises a user-accessible section extending out from the housing; and wherein the rotator is configured to be rotated by a user from the first position to the second position.
23. The sub-assembly according to clause 21, wherein the rotator comprises a proximally directed helical surface extending from a proximal end to a distal end; wherein the distal body of the delivery member guard comprises a distally directed surface configured to move along the proximally directed helical surface of the rotator when the delivery member guard is moved towards the distal end of the housing.
24. The sub-assembly according to clause 21, wherein the sub-assembly comprises a distal button extending out from the distal end of the housing; wherein the distal button is axially movable relative to the housing; wherein the rotator comprises a distally directed helical surface extending from a proximal end of the rotator to a distal end of the rotator; wherein the distal button comprises a button body extending into the housing; wherein the button body comprises a distally directed surface configured to move along the distally directed helical surface of the rotator when the distal button is moved towards the proximal end of the housing.
25. The sub-assembly according to any of clauses 21-24, wherein the rotator comprises a tubular body surrounding the flexible portion of the plunger rod driver when the plunger rod driver is in the distal position.
26. The sub-assembly according to clause 25, wherein the support surface is defined by an inner surface of the tubular body of the rotator.
27. The sub-assembly according to any of clauses 21-26, wherein a cut-out is arranged in a wall of the driver body of the plunger rod driver; wherein the third proximally directed surface of the driver body is next to the cut-out; wherein the rotator comprises a proximally directed arm positioned through the cut-out of the driver body when the plunger rod driver is in the distal position; wherein the proximally directed arm comprises a ledge extending from a proximal end of the proximally directed arm in the direction transverse to the longitudinal axis; and wherein the distally directed surface of the rotator is defined by the ledge.
28. A medicament delivery device comprising the sub-assembly according to any one of the preceding clauses.
29. The medicament delivery device according to clause 28, wherein the medicament delivery device is an injection device.
30. A medicament delivery device comprising the sub-assembly according to clauses 10-13 or any of clauses 13-29 when dependent on clause 10, wherein the medicament delivery device comprises only one spring.

1-15. (canceled)

16. A sub-assembly of a medicament delivery device, the sub-assembly comprising:
a housing extending along a longitudinal axis between a proximal end of the housing and a distal end of the housing;
a delivery member guard telescopically arranged within the proximal end of the housing;
a plunger rod axially movable along the longitudinal axis within the housing, wherein the plunger rod is configured to move into a medicament container; and
a plunger rod driver comprising a driver body having a flexible portion, wherein the flexible portion is positioned between the housing and the plunger rod in a direction transverse to the longitudinal axis, wherein the flexible portion is radially flexible relative to the longitudinal axis,
wherein the plunger rod driver is configured to couple to a power source that is configured to move the plunger rod driver in the direction of the longitudinal axis relative to the housing from a distal position to a proximal position,
wherein the plunger rod driver engages with the plunger rod and does not axially engage with the delivery member guard when the plunger rod driver is in the distal position, and
wherein the flexible portion is configured to be flexed radially outward relative to the longitudinal axis by the plunger rod when the plunger rod driver moves from the distal position to the proximal position so that when the plunger rod driver is in the proximal position, the driver body axially disengages from the plunger rod and axially engages with the delivery member guard.

17. The sub-assembly according to claim 16, wherein the delivery member guard comprises a distally directed surface, wherein the plunger rod comprises a distally directed surface, wherein the flexible portion comprises a first proximally directed surface configured to releasably engage with the distally directed surface of the plunger rod, and wherein the plunger rod driver comprises a second proximally directed surface configured to engage with the distally directed surface of the delivery member guard and a distally directed surface configured to engage with the power source.

18. The sub-assembly according to claim 17, wherein when the plunger rod driver is in the distal position, the first proximally directed surface is adjacent to the distally directed surface of the plunger rod, and the second proximally directed surface is spaced apart from the distally directed surface of the delivery member guard, and wherein when the plunger rod driver is in the proximal position, the first proximally directed surface is disengaged from the distally directed surface of the plunger rod, and the second proximally directed surface is adjacent to the distally directed surface of the delivery member guard.

19. The sub-assembly according to claim 16, wherein the sub-assembly comprises a support surface adjacent to an outer surface of the flexible portion of the driver body in the direction transverse to the longitudinal axis when the driver body is in the proximal position, and wherein the support surface is offset from the outer surface of the flexible portion of the driver body in the direction transverse to the longitudinal axis when the driver body is in the distal position.

20. The sub-assembly according to claim 19, wherein the housing comprises an inner wall extending towards the longitudinal axis, and wherein the support surface is on a surface of the inner wall.

21. The sub-assembly according to claim 19, wherein the flexible portion is radially flexible relative to the longitudinal axis between a relaxed position where the flexible portion flexes radially outward from the longitudinal axis, and a tensioned position where the flexible portion is pressed towards the longitudinal axis by the support surface.

22. The sub-assembly according to claim 16, wherein the flexible portion comprises a pair of flexible arms.

23. The sub-assembly according to claim 22, wherein the pair of flexible arms extends from a fixed, proximal end to a free, distal end in the distal direction of the housing, wherein the first proximally directed surface is arranged at the distal end of the pair of flexible arms.

24. The sub-assembly according to claim 16, wherein the first proximally directed surface is a chamfered surface.

25. The sub-assembly according to claim 24, wherein the power source comprises a spring.

26. The sub-assembly according to claim 25, wherein the spring is a compression spring extending along the longitudinal axis between a proximal end of the spring and a distal end of the spring, wherein the proximal end of the spring is adjacent to the distally directed surface of the driver body, and wherein the distal end of the compression spring is operably connected to the housing.

27. The sub-assembly according to claim 26, wherein the housing comprises a proximally directed surface adjacent to the distal end of the compression spring.

28. The sub-assembly according to claim 16, wherein the driver body comprises a proximal ledge extending from the driver body in the direction transverse to the longitudinal axis, and wherein the distally directed surface of the plunger rod driver is defined by a distally directed surface of the proximal ledge and the second proximally directed surface is defined by a proximally directed surface of the proximal ledge.

29. The sub-assembly according to claim 28, wherein a slot or a groove is arranged in a wall of the housing, wherein the slot of the housing or the groove of the housing extends in the direction of the longitudinal axis from a proximal end to a distal end, and wherein the proximal ledge of the driver body is positioned within the slot of the housing or the groove of the housing.

30. The sub-assembly according to claim 28, wherein the delivery member guard comprises a distal body extending from the tubular proximal body of the delivery member guard towards the distal end of the housing, and wherein a slot is arranged in the distal body, and wherein the proximal ledge of the driver body is positioned within the slot of the delivery member guard.
